(19) United States
(12) Patent Application Publication
(10) Pub. No.: US 2019/0209738 A1
(43) Pub. Date: Jul. 11, 2019

(54) PREPARATION AND APPLICATIONS OF MODIFIED CELLULOSE NANOFIBRILS WITH EXTRACELLULAR MATRIX COMPONENTS AS 3D BIOPRINTING BIOINKS TO CONTROL CELLULAR FATE PROCESSES SUCH AS ADHESION, PROLIFERATION AND DIFFERENTIATION

(71) Applicant: Paul Gatenholm, Riner, VA (US)

(72) Inventor: Paul Gatenholm, Riner, VA (US)

(21) Appl. No.: 16/307,852

(22) PCT Filed: Jun. 9, 2017
(86) PCT No.: PCT/US17/36895

§ 371 Date: Dec. 6, 2018

Related U.S. Application Data
(60) Provisional application No. 62/347,950, filed on Jun. 9, 2016.

(51) Int. Cl. A61L 27/60; A61K 35/36; C12N 5/0793; C12Q 1/02; C12N 5/071; C12N 5/077; A61L 27/24; A61L 27/36; A61P 25/28; A61P 25/16

(52) U.S. Cl. CPC A61L 27/60; C12N 2533/74; C12N 5/0619; C12Q 1/025; C12N 5/0629; C12N 5/0656; A61L 27/24; A61L 27/362; A61P 25/28; A61P 25/16; C12N 2533/78; C12N 2533/90; C12N 2533/54; C12N 2533/52; C12N 2501/15; C12N 2501/155; A61L 2430/40; C12N 2533/80; A61K 35/36

(57) ABSTRACT

The present invention relates to modification of cellulose nanofibrils (CNF) with extracellular matrix components such as collagen, elastin, fibronectin or RGD sequences or growth factors such as TGFBeta using for example EDS-NHS conjugation method and preparation of bioinks for 3D Bioprinting of tissue models such as human skin or neural tissue. Cellulose nanofibrils provide excellent printing fidelity which is crucial for diffusion of oxygen and diffusion of nutrients into the 3D bioprinted constructs. The surface conjugated extracellular matrix components induce biological activity by providing adhesion sites or inducing differentiation process. 3D Bioprinted bioinks based on CNF bioinks showed great ability inducing adhesion of human fibroblasts and stimulating Collagen I production. Such bioinks are thus suitable for 3D Bioprinting of tissue models.

### BACKGROUND OF THE INVENTION

### Field of the Invention

The present invention relates to materials based on cellulose nanofibrils modified with extracellular matrix (ECM) components such as collagen, elastin, fibronectin or peptide motifs such as RGD or GRGDSP, laminin or growth factors such as TGFBeta or BMP2 or BMP7 and their use as 3D Bioprinting bioinks to control cellular fate processes such as adhesion, proliferation and/or differentiation. The bioinks based on modified cellulose nanofibrils may be used for 3D Bioprinting processes using human or animal cells. The advantage of the modification of nanocellulose with extracellular matrix components, which interact with integrins at the cell surface level, is control of cell fate processes. The modified cellulose nanofibrils with ECM behave like cell instructive biomaterials. When non-modified cellulose nanofibrils (CNF) which are bioinert are used together with cells there is often a lack of cell adhesion affecting cellular fate processes and resulting in cell death. In contrast, CNF modified with molecules which can communicate with cells by, for example, providing adhesion sites for cell surface bounded integrins, result in good cell viability and enhanced proliferation. In addition, the cell instructions can be provided to initiate the differentiation process so the stem cells become, for example, chondrocytes or osteoblasts. In one aspect of the present invention, modification of cellulose fibrils is carried out in aqueous medium and does not affect the colloidal stability of CNF. The modified CNF can be used as such or can be mixed with non-modified CNF to produce bioinks for 3D Bioprinting. It is beneficial according to the invention herein to use the second component in such bioinks which can provide crosslinking. Such a component may be tyramine conjugated hyaluronic acid, which, after addition of horse radish peroxidase and hydrogen peroxide, is crosslinked with covalent bonding. Another example of a crosslinkable component is alginate, which crosslinks upon addition of calcium chloride. Another component can be fibrinogen, which crosslinks upon addition of thrombin. Another component can be gelatin or collagen modified with UV crosslinkable groups, and crosslinking is provided by UV. The bioinks described in this invention can be mixed with cells and 3D Bioprinted. Good printing fidelity is achieved under the current invention, because shear thinning properties of CNF are advantageous for decreasing viscosity under high shear rates, which results in printed constructs with high porosity. That is crucial for culturing of cells in vitro, in a bioreactor, or for implantation in animals and/or humans because the porous structures provide good diffusion of oxygen and nutrients. The bioinks described in this invention have shown attachment of human fibroblasts at several points resulting in cell stretching and enhanced Collagen I production. This is of importance for growing skin for implantation or growing skin-like models for testing cosmetics, health care products, or drugs. Another application of this invention is adhesion of neural cells which is crucial for formation of a neural network that may be used to repair damaged nerves or as models to study diseases such as Alzheimer's or Parkinsons. Another application is to control viability, proliferation, and induce differentiation of stem cells. Stem cells can be derived from bone marrow (Mesenchymal Stem Cells, MSC) or derived from adipose tissue (Adipose Stem Cells, ASC) or induced pluripotent stem cells (iPSC) can be used. The bioinks described in this invention can affect the stem cell differentiation through interactions with conjugated growth factors such as TGFBeta or BMP or adhesion molecules such laminin. Cellulose nanofibrils of different origins are covered by this invention. They can originate from wood, primary cell wall, be produced by bacteria, or isolated from tunicates.

### Description of Related Art

3D Bioprinting is an emerging technology which can provide solutions to many problems related to health. 3D Bioprinting can potentially replicate any tissue or organ by building biological material layer by layer. 3D Bioprinting requires a 3D bioprinter which can deposit cells with high resolution and also can add signaling molecules. But cells cannot be deposited alone. They need supporting material which is called bioink. The function of bioink is to facilitate viable cell deposition in a predetermined pattern and then become the scaffold when cells are cultured in vitro or in vivo. Among the most important properties of bioinks are rheological properties. All polymer solutions are shear thinning which means that the viscosity is decreased with increased shear rate. Cellulose nanofibrils which can be produced by bacteria or isolated from primary or secondary cell walls of plants are 8-10 nm in diameter and can be up to a micrometer long. They are hydrophilic and therefore bind water on their surfaces. They form hydrogels at low solid content (1-2%). CNF are extremely shear thinning and have high zero shear viscosity. The hydrophilic nature of the CNF surfaces covered by water prevent them from protein adsorption and make them bioinert. Cells do not recognize CNF surfaces which is an advantage, as taught herein, when it comes to biocompatibility since there is no foreign body reaction. But, because they are bioinert, they do not facilitate cell attachment. As disclosed herein, many types of cells need to be attached to a surface or to a network of extracellular matrix components to migrate, proliferate, differentiate, produce extracellular matrix and become tissue. The extracellular matrix components which provide cell attachment according to the present invention are collagen, elastin, fibronectin and laminin. Another group of important components of extracellular matrix which affect cellular processes are growth factors such as TGFBeta and Bone Morphogenic Protein (BMP2 or BMP7). FIG. 1 illustrates how different ECM components can be added through bioconjugation processes onto a cellulose backbone. As explained in this application, they stimulate cell proliferation and also induce cell differentiation. The extracellular matrix components can be added to the bioink but they are easily washed out during change of the medium or diffuse out in in vivo conditions. It is therefore advantageous to bind them to the network of CNF in the bioink. In this way the unique rheological properties of CNF which provide printing fidelity are combined with desired biological properties to control cellular functions and promote tissue formation. The bioinks based on CNF conjugated with ECM components can behave as cell instructive biomaterials.

There are different methods to chemically modify CNF (conjugate or bioconjugate when it comes to the biological molecule). The accessibility of CNF for bioconjugation is determined by the hydroxylic content of cellulose backbone. Several compounds can turn the hydroxyl residues into intermediate reactive derivatives having suitable leaving groups for nucleophilic substitution. The most common activating agents for cellulose are N-hydroxysuccinimide esters, carbonyldiimidazole, epoxide compounds, sodium periodate, tresyl- and tosyl-chloride, cyanogen bromide, cyanuric chloride, as well and several chloroformate derivatives. The activation process requires, however, non aqueous solutions such as dry dioxane, acetone, THF, DMF or DMSO to prevent hydrolysis of the reactive intermediate products in aqueous solution. Hydroxyl groups can be modified in an aqueous environment with anhydrides, chloroacetic acid or radical-mediated oxidation with (2,2,6,6-tetramethylpiperidin-1-yl)oxidanyl to produce carboxylate functionality for further conjugation purposes with the use of carbodiimides as crosslinkers (1). In this application, for the conjugation of ECM components, the carboxylic acid on cellulose in a carbodiimide reaction with 1-Ethyl-3-(3-dimethylaminopropyl)carbodiimide (EDC) and N-hydroxysulfosuccinimide (NHS) was used. Available CNF contains carboxylic groups, which are introduced prior to a homogenization process in which cellulose nanofibrils are produced. Carboxylation can also be performed using, for example, TEMPO reaction. This three-step reaction starts the reaction with the carbodiimide unit, which is the most crucial step in the reaction. The EDC reacts with the carboxyl acid, creating an active O-acylisourea intermediate. This can directly react with a primary amine, but proceeding with addition of NHS forms a more stable NHS ester. The NHS ester also reacts well with primary amines, but has the benefit of performing the coupling at physiological pH. Proceeding with NHS addition also improves the yield. Regulation of pH through the reaction should also be done to further increase the yield. The diimide coupling happens more rapidly at pH 5.3-5.5 and starting the reaction at this pH range is desired. As stated previously, NHS guided amide formation can be done at physiological pH and regulating the pH back should be done as it otherwise could influence the conformation of proteins. FIG. 2 shows schematically the reaction conditions employed in this invention for bioconjugation of ECM components to CNF.

### SUMMARY OF THE INVENTION

This invention describes preparation of conjugated cellulose nanofibrils with extracellular matrix components such as collagen, elastin, fibronectin or RGD peptides which represent fibronectin, and with adhesive components such as laminin and with growth factors such as TGFBeta and BMP2 or BMP7. These conjugated components promote cell adhesion, increase cell viability and cell proliferation and promote cell differentiation. In this invention, human dermal fibroblasts were shown to strongly attach to CNF conjugated with fibronectin and RGD peptides. The attachment resulted in cell stretching which induced collagen I production. Another modification which is described in this invention is binding of TGFBeta to CNF. TGFBeta conjugated CNF are shown herein to stimulate proliferation of stem cells, including mesenchymal stem cells, and cell differentiation towards chondrocytes. In another example, this application teaches conjugated CNF with laminin 521, which showed differentiation of iPS cells towards chondrocytes. EDS-NHS conjugation in this application has been used for binding of extracellular matrix components. Other conjugation methods can be used instead.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 shows schematically modification of cellulose nanofibrils with extracellular matrix components, proteins or peptides.

FIG. 2 shows schematic reaction of bioconjugation of cellulose nanofibrils with extracellular matrix components (ECM), proteins or peptides.

FIG. 3 shows fibroblasts-laden bioink constructs with printing fidelity. This is important according to the present invention for transport of nutrients and oxygen to the cells in the construct.

FIG. 4 shows cell viability in a printed construct with RGD-modified nanocellulose. Green spots represent cells which are alive and red spots represent the dead cells. The cell viability is more than 80% in this example.

FIG. 5 shows cell morphology in printed constructs after 1 and 7 days culturing. Green spots represent cytoskeleton and blue spots represent cell nuclei.

a) Unmodified nanocellulose fibrils bioink 1 day

b) RGD-modified nanocellulose fibrils 1 day

c) RGD-modified nanocellulose fibrils 7 days

FIG. 6 shows iPSC viability in laminin 521 bioconjugated nanocellulose bioink.

FIG. 7 shows the effect of laminin 521 bioconjugated nanocellulose bioink on iPSC differentiation.

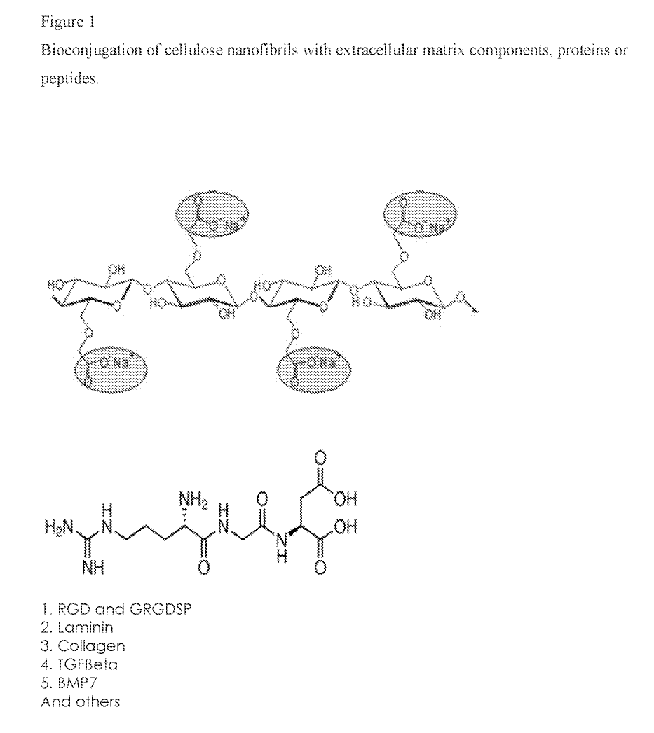

### DETAILED DESCRIPTION OF VARIOUS EMBODIMENTS OF THE INVENTION

To facilitate a better understanding of the present invention, the following examples of certain aspects of some embodiments are given. In no way should the following examples be read to limit, or define, the scope of the invention.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

### Example 1

Bioconjugation with RGD Peptides and 3D Bioprinting of Skin-Like Model

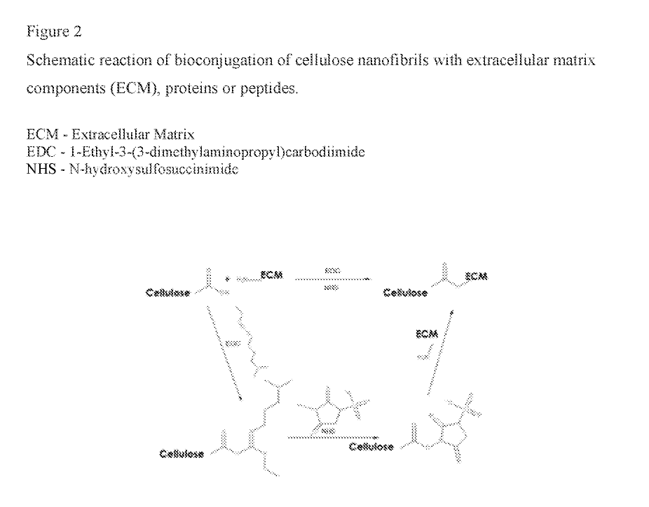

Cellulose nanofibrils which were carboxymethylated were modified using EDS-NHS conjugation method with RGD peptides. Afterwards, 24 reaction CNF were placed in dialysis tubing with cut-off 10 kD for two weeks. Purified conjugated CNF was mixed with non-modified CNF used for bioink preparation. Two different bioinks were prepared. The first bioink was composed of RGD-CNF and alginate which provided crosslinking after addition of calcium chloride. The second bioink was prepared by addition of Tyramine modified hyaluronic acid and crosslinked with horse radish peroxidase and hydrogen peroxide. Both bioinks had good printability. 6 million primary human fibroblasts passage #3 were thawed and seeded into two 150 cm2 T-flasks. When the culture reached approximately 90% confluence, the cells were harvested using TrypLE and the flask was gently tapped to make the cells detach from the surface. The cells were counted (1.9 M cells/mL) with Tryphan-blue staining and the cell viability was calculated to ensure the cells were alive. The cells were then centrifuged and resuspended in medium and then seeded with 2,500 cells/cm2 into a T150 flask. The medium (DMEM, 1% GlutaMAX with 10% FBS and 1% Pen/Strep with phenol red) was changed three times per week. The cells were mixed with the bioinks to provide a final concentration of 5.2 million cells/ml and then carefully moved into the printer cartridge. Constructs were printed in a grid pattern in three layers with the dimensions of 6 mm×6 mm×1 mm (pressure: 24 kPa, feed rate: 10 mm/s) using the 3D-bioprinter INKREDIBLE from CELLINK AB, Sweden (see FIG. 2). After printing, the constructs were crosslinked.

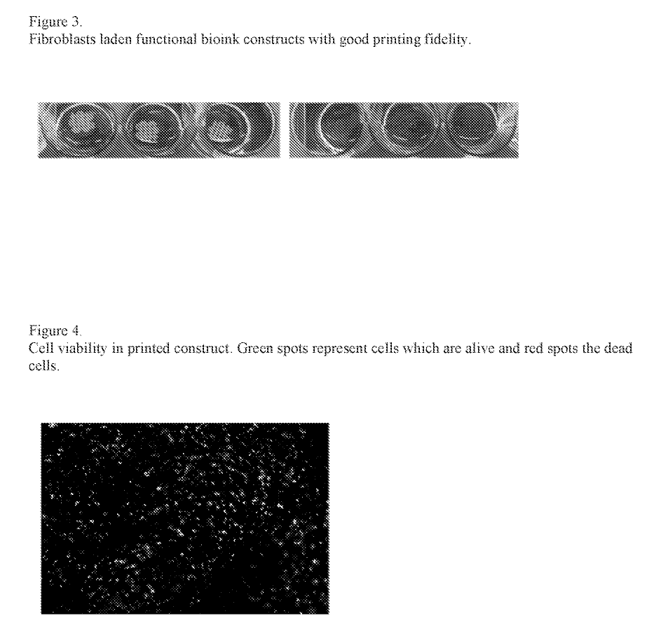

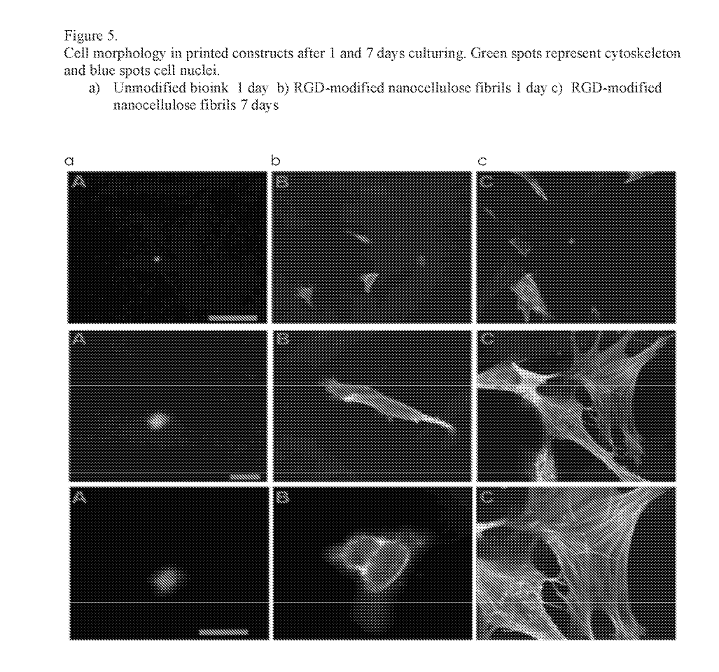

The constructs were cultured statically for 14 days in an incubator at 37° C. and the medium was changed every third day. TGFBeta was added at a concentration of five ng/ml medium to some of the constructs. The constructs were analyzed for cell viability, morphology and collagen production after 14 days. Live/Dead staining was performed on three constructs from each bioink of the static culture on day 1, day 7, and day 14 using a LIVE/DEAD Cell Imaging Kit (R37601 Life Technologies). FIG. 3 shows good cell viability (more than 70%) for all printed constructs. On day 1 and day 7, the static culture constructs were imaged using a confocal microscope. The FITC was used to visualize the cytoskeleton (green) and the DAPI was used to visualize the nuclei (blue) of the cells. Images were taken at 4×, 10×, and 20× magnification to analyze cell morphology. ImageJ was used to overlay images of the cytoskeletons and nuclei. FIG. 4 a) shows the morphology of fibroblasts in non-modified CNF bioink. The cells were round and not stretched at all. FIG. 4 b) shows fibroblasts in RGD-modified CNF bioink with alginate after 1 day. The cells were stretched because they were able to attach to RGD peptides which were conjugated with CNF. FIG. 4 c) shows fibroblasts in RGD-modified CNF bioink with alginate after 7 days culturing. There is an important effect due to the current invention, which is seen in increased cell proliferation and continued stretching. These effects were not seen for the cells printed with bioink which was not modified with RGD. The constructs were analyzed with PCR and the constructs with RGD-modified CNF showed upregulated genes for production of Collagen I.

### Example 2

Bioconjugation Reaction Between Nanocellulose Fibrils and Laminin 521 and 3D Bioprinting with iPSC

The cellulose-ECM conjugates were prepared using a carbodiimide coupling method. Carboxymethylated CNFs, MFC8 (3 wt %) (Stora Enso, Finland) was diluted in MiliQ water (0.2 wt %) and mixed at 10,000 rpm with ultraturrax for ten minutes. Reaction was carried out with 1-Ethyl-3-(3-dimethylaminopropyl)carbodiimide, EDC (Sigma Aldrich) and N-hydroxysulfosuccinimide, NHS (Sigma Aldrich) in excess to activate all carboxyl groups on the cellulose nanofibrils; pH was adjusted with HCl to a desired 5.3. Addition of ECM such as laminin 521 (Biolamina, Sweden) in different weight ratios of dry cellulose mass to laminin was then performed, followed by pH regulation to pH 7.2 and the reaction was put on ice and run for 24 hours.

The dispersion was dialyzed for five days in MilliQ water using membrane with a cut-off of 10 kD. The dialysis water was refreshed two times a day. Samples were then centrifuged at 12,000 rpm for ten minutes. The supernatant was separated from the concentrated gel. The CNF-Laminin gel was sterilized in an electron beam at 25 kGy (Herotron, Germany) before mixing with other ink components. To improve printability, the sterilized samples were centrifuged at 4,000 rpm for ten minutes. To maintain physiological osmolarity, 4.6% mannitol (Sigma Aldrich) was added to the hydrogel solution.

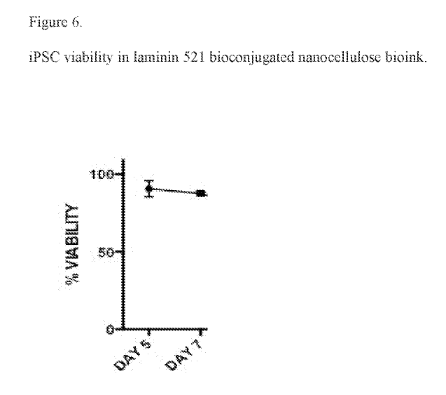

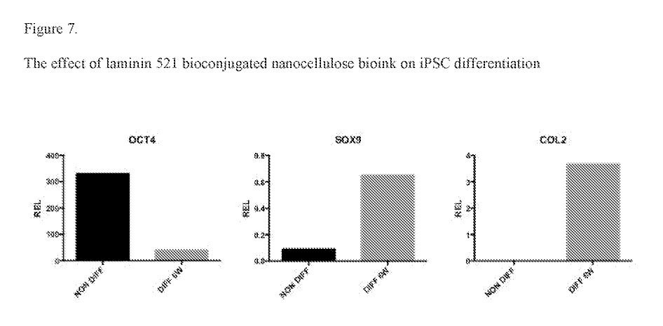

The cells were mixed with bioinks, in a mixing process by connecting syringes with each liquid and, through a back and forth motion, mixing was achieved. This procedure was performed for at least five cycles, and any color variations in the ink resulted in further mixing. 3D bioprinting with cell-ink mixtures was performed with a 3D Bioprinter INKREDIBLE (Cellink AB, Sweden), sterilized using 70% ethanol and kept in a sterile LAF bench during all printing to eliminate contamination. Printing was performed in ambient temperature and humidity. Post printing crosslinking was performed by addition of CaCl₂ 0.1M (Sigma Aldrich) and allowed to crosslink over five minutes. CaCl₂ was then replaced with cell culture medium and plates were placed into an incubator at 37° CO₂ 5%, with medium exchanged every second day. iPSC lines were generated from surplus chondrocytes using mRNA-based reprogramming. The A2B iPSC line was maintained under feeder-free conditions in Cellartis DEF-CS™ (TaKaRa ClonTech, Sweden). This iPSC line was karyotype-tested, was normal even at late passages, was pluripotent with regards to the expression of pluripotency markers, and was able to differentiate into all germ layers. This line was also shown to be superior in the differentiation protocol to generate articular cartilage matrix in 3D pellets and was used for 3D printing in subsequent experiments. In addition, iPSC-conditioned DEF medium from confluent clone A2B iPSCs was used after printing since increased survival had been noticed for single cells in a conditioned medium. For co-culture conditions, human surplus chondrocytes were irradiated (iChons) before being mixed with iPSCs to prevent the proliferation of the chondrocytes. The cell number was counted in a nucleocounter NC-200™ using Vial-Casettes™ (ChemoMetec, Denmark). iPS cells were tested for pluripotency after printing and at day 8 the differentiation protocol was introduced to convert the iPS cells into chondrocytes. These cells are found in the cartilage in the body where they produce collagen II, the main protein in cartilage. It is expected to see viability and cell count go down starting a differentiation protocol. However, according to the present invention, high viability and high proliferation rates start pre-differentiation, indicating that the cells prefer the currently claimed conjugated ink, as compared to previously released data and inks using unmodified CNF. (See FIG. 6, for example). Pluripotency after printing and the differentiation into chondrocytes was analyzed by pCR and by looking at the gene expression of OCT4 (pluripotency marker), SOX9 (marker of protein during chondrocyte differentiation), and COL2 (gene for instruction of collagen II production), according to FIG. 7. pCR analysis showed that the cells were still pluripotent after printing as shown in the OCT4 response. After six weeks of differentiation, most cells had lost their pluripotency, deducted by an OCT4 decrease. This is important because remaining pluripotent cells in a clinical setting have the potential for tumor growth. The present invention also helps to conclude that genes SOX9 and COL2 have been turned on, factors required during chondrocyte differentiation. In conclusion, laminin 521 conjugated CNF bioink provide excellent cell viability and promote cell differentiation, according to the studies and inventive processes/products claimed herein.

### Example 3

Bioconjugation with TGFBeta1 and 3D Bioprinting of Cartilage Tissue with Stem Cells

The cellulose-TGFBeta1 (TGFB1) conjugates were prepared using a carbodiimide coupling method. Carboxymethylated CNFs, MFC8 (3 wt %) (Stora Enso, Finland) were diluted in MilliQ water (0.2 wt %) and mixed at 10,000 rpm with ultraturrax for ten minutes. Reaction was carried out with 1-Ethyl-3-(3-dimethylaminopropyl)carbodiimide, EDC (Sigma Aldrich) and N-hydroxysulfosuccinimide, NHS (Sigma Aldrich) in excess to activate all carboxyl groups on the cellulose nanofibrils; pH was adjusted with HCl to a desired 5.3. Addition of ECM such as TGFBeta1 (Termofisher, Sweden) in different weight ratios of dry cellulose mass to TGFBeta1 was then performed, followed by pH regulation to pH 7.2 and the reaction was put on ice and run for 24 hours.

The dispersion was dialyzed for five days in MilliQ water using membrane with a cut-off of 10 kD. The dialysis water was refreshed two times a day. Samples were then centrifuged at 12,000 rpm for ten minutes. The supernatant was separated from the concentrated gel. The bioink with 3% dry matter containing CNF (60%), conjugated CNF with TGFB1 (20%) was prepared and sterilized in electron beam at 25 kGy (Herotron, Germany) before mixing with other ink components. One example of crosslinkable component was alginate SLG100 from Nova Matrix Norway (20%). To improve printability, the sterilized samples were centrifuged at 4,000 rpm for ten minutes. To maintain physiological osmolarity, 4.6% mannitol (Sigma Aldrich) was added to the hydrogel solution.

Human nasoseptal cartilage biopsies were obtained during routine surgeries at the Department of Otorhinolaryngology, University Medical Center Ulm, Germany. Cartilage harvesting was approved by the University of Ulm Ethics Committee (No. 152/08), and patients involved in this study agreed to the Informed Consent. Donor age ranged from 22 to 54 years, with an average age of 34. All cartilage samples were first rinsed in standard culture medium DMEM/Ham's F-12(1:1, Biochrom), supplemented with fetal bovine serum (FBS, 10%; Biochrom) and 1% penicillin-streptomycin, under sterile conditions. Adherent non-cartilaginous tissues, such as perichondrium or epithelium, were removed. To isolate human primary nasal chondrocytes (hNC), the cartilage samples were rinsed in standard culture medium, minced, transferred to digestion medium (standard culture medium without FBS, containing 0.3% collagenase type II; Worthington), and incubated for 16 hours at 37° C. in a shaking water bath. After centrifugation, the total cell number and viability were determined by trypan blue exclusion method. Subsequently, hNCs were seeded for expansion with an initial density of 5×10³ cells cm². When reaching 80-90% confluence, cells were detached, counted, and cryopreserved to ensure an equal treatment for all hNCs harvested from different patients. Cryopreserved hNCs were thawed and expanded once in monolayer. When reaching 80-90% confluence, cells were detached, counted and resuspended in culture medium, before mixing with the Adipose derived stem cells (ASC) and bioink. HNCs (30×10⁶ cells) were resuspended in 200 mL of culture medium per mL of bioink and after centrifugation were mixed with ASC (female donor, cells purchased from RoosterBio, USA) at ratio hNC:ASC 20:80 with the CNF bioink to obtain a final concentration of 10×10⁶ cells/mL of bioink. The cell-laden hydrogel was mixed with a microspatula, until a homogeneous pink color was achieved and subsequently loaded into a printer-compatible cartridge. 3D bioprinting with cell-laden bioinks was performed with 3D Bioprinter INKREDIBLE (Cellink AB, Sweden), sterilized using 70% ethanol and kept in sterile LAF bench during all printing to eliminate contamination. Printing was performed in ambient temperature and humidity. Grids with size 6×6×1 mm, 2 layers were printed using a 410 μm nozzle. Post printing crosslinking was performed by addition of CaCl₂ 0.1M (Sigma Aldrich) and allowed to crosslink over five minutes. CaCl₂ was then replaced with cell culture medium and plates were placed into incubator 37° CO₂ 5%, with medium exchanged every second day. Reduced chondrogenic and differentiation media, with and without TGFB1 were used for culturing. TGFB1 conjugated bioink showed good printability, good cell viability (more than 85%) and enhanced chondrocytes proliferation. ACS cells were differentiated towards chondrocytes after 21 days of culturing as determined by production of extracellular matrix components such as Collagen 2 and proteoglycans.

### Example 4

3D Bioprinting of Neural Tissue

CNF modified with laminin was used to prepare bioink with addition of carbon nanotubes. Such conductive bioink showed cell adhesion and formation of a neural network.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

### REFERENCES (INCORPORATED HEREIN BY REFERENCE)

1. Kuzmenko, V., S. Saemfors, D. Haegg, and P. Gatenholm, Universal method for protein bioconjugation with nanocellulose scaffolds for increased cell adhesion. Mater. Sci. Eng., C, 2013. 33(8): p. 4599-4607.

1-3. (canceled)

4. A bioink comprising:
cellulose nanofibrils (CNF) modified with one or more extracellular matrix components.

5. The bioink of claim 4, with or without cells.

6-7. (canceled)

8. The bioink of claim 5, further comprising alginate.

9. The bioink of claim 5, further comprising hyaluronic acid.

10. A 3D Bioprinted tissue comprising fibroblasts in cellulose nanofibrils (CNF) modified with extracellular matrix components.

11. The 3D Bioprinted tissue of claim 10, wherein space between bioink printed grids allows diffusion of nutrients, oxygen, proteins, and/or growth factors.

12. (canceled)

13. The 3D Bioprinted tissue of claim 10, wherein the fibroblasts are stimulated by TGFBeta.

14. The 3D Bioprinted tissue of claim 13, wherein the fibroblasts are present in combination with the cellulose nanofibrils (CNF) modified with extracellular matrix components.

15. The 3D Bioprinted tissue of claim 10, wherein the cellulose nanofibrils (CNF) are modified with one or more extracellular matrix components chosen from collagen, elastin, fibronectin or RGD sequences, laminin, growth factors TGFBeta, or Bone Morphogenic Protein.

16. (canceled)

17. The 3D Bioprinted tissue of claim 10, which is neural tissue or dermis tissue.

18-21. (canceled)

22. A method of treating animals and/or humans which suffer from tissue defect by implantation of 3D Bioprinted tissue comprising cellulose nanofibrils (CNF) modified with extracellular matrix components.

23-24. (canceled)

25. The bioink of claim 5, further comprising gelatin or collagen modified with UV crosslinkable groups.

26-27. (canceled)

28. The method of treating animals and/or humans of claim 22, wherein the tissue defect is due to Alzheimer's or Parkinsons disease.

29-33. (canceled)

34. The bioink of claim 5, wherein the cellulose nanofibrils (CNF) are modified with one or more extracellular matrix components chosen from collagen, elastin, fibronectin or RGD sequences, laminin, growth factors, TGFBeta, or Bone Morphogenic Protein.
